(19) United States
(12) Patent Application Publication
(10) Pub. No.: US 2003/0073959 A1
(43) Pub. Date: Apr. 17, 2003

(54) Syringes

(76) Inventor: Marc Koska, West Sussex (GB)

Correspondence Address: MICHAEL BEST & FRIEDRICH LLC, 401 NORTH MICHIGAN AVENUE, SUITE 1700, CHICAGO, IL, 60611-4212, US

(21) Appl. No.: 10/148,938

(22) PCT Filed: Dec. 4, 2000
(86) PCT No.: PCT/GB00/04636

(51) Int. Cl. A61M 5/00

(52) U.S. Cl. 604/240

(57) ABSTRACT

A syringe barrel 1 has a hollow distal projection 7 to which a needle 10 is attached. A hub 11 is used to attach the needle 10 to the barrel 1. The hub 11 has a distal part 12 to which the needle 10 is fixed, and a proximal part 14 for attachment to the barrel 1. The proximal part 14 includes a bore 15 for receiving the projection 7 and an outwardly-extending flange 16. The syringe barrel 1 has a collar 9 surrounding and spaced from the projection 7 to define a space for receiving the proximal part 14 of the hub 11. The internal surface of the collar 9 includes a single outwardly-extending locking projection 19, arranged so that the flange 16 engages its proximal side to lock the hub 11 to the barrel 1. The locking projection 19 has a shallow lead-in ramp 20 on the distal side, a more steeply-angled ramp 21 on the proximal side, and a cylindrical surface 22 separating the ramps 20, 21. The projection 19 fixes the hub 11 to the barrel 1 so it is difficult, if not impossible, to remove.

This invention relates to syringes, and in particular to means for attaching a needle to a syringe.

A syringe barrel normally has at its distal end a hollow open projection to which the injection needle is attached.

It is known for the needle to be inserted into a passage in the end of the projection and glued in place to provide permanent fixing of the needle to the barrel. Permanent fixing is advantageous in use, as the needle cannot be removed accidentally or deliberately, but this construction has disadvantages for manufacture. If different lengths or types of needle are needed on the same barrel the apparatus used to attach the needle must be changed, which is expensive and time-consuming in manufacture.

In an alternative known arrangement, the needle is glued to a distal part of a separate hub, which has a proximal part attached to the syringe barrel. This makes it easy to provide different needles on the same barrel, so reducing manufacturing costs. The most common construction is a luer arrangement, in which the barrel projection has a slightly conical outer surface (tapering away from the barrel), while the proximal part of the hub has a complementary conical bore for receiving the projection. In a luer slip arrangement the hub is a friction fit on the projection. This is intended to be a permanent fixing, but the hub can be blown off the projection during injection by the pressure of the liquid in the barrel. In a luer lock arrangement the hub has a screw-threaded engagement with the barrel, so that either the hub and the projection have complementary screw threads, or a flange on the hub co-operates with a screw thread formed on the internal surface of an annular collar surrounding the projection. This may prevent the hub being blown off during injection, but of course is not a permanent fixing, as the hub can simply be removed by unscrewing it. Providing the screw threads may increase manufacturing costs.

In another type of luer lock arrangement the barrel has a collar surrounding and spaced from the projection to define a space for receiving the proximal part of the hub. The internal surface of the collar has one or more inwardly-projecting ribs, past which a flange on the hub can snap into position. In U.S. Pat. No. 3,179,107 the collar has a single rounded rib, with a gentle slope on its distal side and a sharper shoulder on its proximal side. This enables the hub to be attached easily and held firmly, but is still intended to allow removal of the hub. In U.S. Pat. No. 2,902,995 the collar has several sawtooth rings, which are intended to provide a more permanent fixing. However, this arrangement is more expensive to manufacture, and because of the plurality of engaging positions, it is not easy to ensure that the luer arrangement is properly engaged.

According to the present invention, a means for attaching a needle to a syringe barrel having a hollow distal projection comprises a hub having a distal part to which the needle is fixed and a proximal part for attachment to the syringe barrel and including a bore for receiving the projection and an outwardly-extending flange means, the syringe barrel having collar means surrounding and spaced from the projection to define a space for receiving the proximal part of the hub, the internal surface of the collar means including a single inwardly-extending locking projection, the flange means being adapted to engage the proximal side of the locking projection to lock the hub to the barrel, and the locking projection comprising a shallow lead-in ramp on the distal side, a more steeply-angled ramp on the proximal side, and a cylindrical surface separating the ramps.

Thus, the hub is attached to the barrel by a simple pushing movement which moves the flange means past the locking projection so that the flange means engages behind it, locking the hub to the barrel, and making it difficult if not impossible to remove. The shallow lead-in ramp assists the insertion of the hub, while the steeper angle of the locking ramp assists the locking arrangement. The locking arrangement ensures that the hub cannot be blown off during injection, or removed accidentally or deliberately. A permanent fixing is therefore achieved easily, but with the reduced manufacturing costs associated with the use of a hub. The flange means and locking projection are easier to manufacture than screw threads.

Preferably the barrel projection and the hub have a luer construction.

The flange means is preferably arranged at the proximal end of the hub. The flange means may have a circular perimeter, or have one or more projections to assist the locking engagement.

Conveniently the collar means is annular. The locking projection preferably extends continuously round the surface of the collar to secure the locking engagement. It is preferably located close to the proximal end of the collar means, to ensure that, in the locked position, the hub is fully engaged on the projection.

An embodiment of the invention is illustrated by way of example only in the accompanying drawings in which:

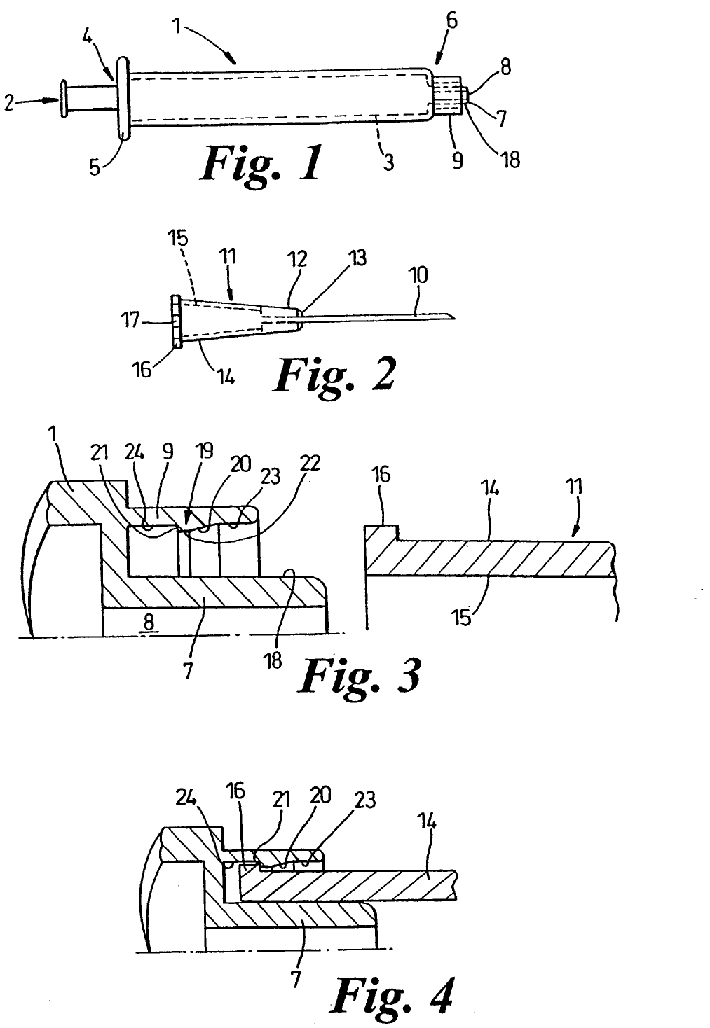

FIG. 1 is a side elevation of a syringe;

FIG. 2 is a side elevation of a needle attached to a hub;

FIG. 3 is a partial cross-section through the distal projection on the syringe barrel and part of the hub before assembly; and

FIG. 4 is a partial cross section similar to FIG. 3 and showing the hub assembled onto the barrel projection.

The syringe shown in FIG. 1 comprises a barrel 1 in which a plunger 2 works. The barrel 1 is injection-moulded from plastics and has a cylindrical inner surface 3, an open proximal end 4 provided with a gripping flange 5, and a distal end 6 with a hollow distal projection 7 having a liquid outlet 8. An annular collar 9 surrounds the projection 7 and is spaced from it. A needle 10 (see FIG. 2) is attached to the projection 7 by means of a hub 11.

The hub 11 is open-ended, hollow and injection-moulded from plastics. A distal part 12 has a bore of small diameter to receive the hypodermic needle 10. The needle 10 is glued in place by adhesive 13. A proximal part 14 of the hub 11 is conical, with a conical bore 15 tapering towards the needle 10. The bore 15 is adapted to receive the barrel projection 7. The proximal part 14 terminates in a flange 16, which is substantially circular, with a pair of opposing projections 17.

The barrel projection 7 has an external surface 18 which is also conical, and tapers away from the barrel 1. The surface 18 is complementary to the bore 15 on the hub 11, to form a luer slip arrangement with a friction fit. Typically, the angle of the cone is about 6°.

As best seen in FIGS. 3 and 4 the projection 7 projects beyond the annular collar 9. The collar 9 has an inwardly-extending locking projection 19 which extends continuously round the internal surface of the collar 9. The locking projection 19 is substantially triangular in section. It has a shallow lead-in ramp 20 on the distal side, and a more steeply-angled locking ramp 21 on the proximal side. As shown, the two ramps 20, 21 are separated by a short cylindrical section 22. The angle between the locking ramp 21 and the internal surface of the collar 9 is about 155°. The internal surface has cylindrical distal and proximal portions 23, 24 respectively of the same diameter. For assembly of the hub 11 into the projection 7, in order to attach the needle 10 to the barrel 1, the hub 11 is placed onto the projection 7, so that the projection 7 enters the bore 15, and the flange 16 of the proximal part 14 enters the annular space between the projection 7 and the collar 9. The outer diameter of the flange 16 is slightly smaller than the diameter of the distal portion 23.

As the hub 11 is pushed towards the barrel 1 the luer connection is made, and the flange 16 rides along the lead-in ramp 20 and the section 22, deflecting the collar 9 resiliently outwardly. At the end of the section 22 the flange 16 rides along the locking ramp 21, and reaches the proximal portion 23 of the collar 9, where the internal surface is again cylindrical and slightly larger than the diameter of the flange 16. This enables the collar 9 to return to its original position, and the engagement of the flange 16 with the locking ramp 21 serves to lock the hub 11 to the barrel 1. The projections 17 assist in the locking engagement. The angle of the locking ramp 21 is chosen so that it is extremely difficult, if not impossible, for the flange 16 to return past the locking projection 19. The hub 11 and needle 10 are therefore permanently fixed to the syringe.

1. A means for attaching a needle to a syringe barrel having a hollow distal projection, said means comprising a hub having a distal part to which said needle is fixed and a proximal part for attachment to said syringe barrel, said proximal part including a bore for receiving said projection and an outwardly-extending flange means, said syringe barrel having collar means surrounding and spaced from said projection to define a space for receiving said proximal part of said hub, the internal surface of said collar means including a single inwardly-extending locking projection, said flange means being adapted to engage the proximal side of said locking projection to lock said hub to said barrel, wherein said locking projection comprises a shallow lead-in ramp on the distal side, a more steeply-angled ramp on the proximal side, and a cylindrical surface separating said ramps.

2. An attachment means as claimed in claim 1, wherein said barrel projection and said hub have a luer construction.

3. An attachment means as claimed in claim 1, wherein said flange means is arranged at the proximal end of said hub.

4. An attachment means as claimed in claim 1, wherein said flange means has a circular perimeter.

5. An attachment means as claimed in claim 1, wherein said flange means has one or more projections to assist the locking engagement.

6. An attachment means as claimed in any claim 1, wherein said collar means is annular.

7. An attachment means as claimed in claim 1, wherein said locking projection extends continuously round the surface of said collar means to secure the locking engagement.

8. An attachment means as claimed in claim 1, wherein said locking projection is located close to the proximal end of said collar means.
